(19) United States
(12) Patent Application Publication
(10) Pub. No.: US 2013/0164830 A1
(43) Pub. Date: Jun. 27, 2013

(54) Combination Assay Device and Method for Detecting Compounds in Vaginal Fluid

(71) Applicant: Kimberly-Clark Worldwide, Inc., Neenah, WI (US)

(72) Inventors: Ronnie Lee Phillips, Atlanta, GA (US); Stephanie Michelle Martin, Johns Creek, GA (US)

(73) Assignee: KIMBERLY-CLARK WORLDWIDE, INC., Neenah, WI (US)

(21) Appl. No.: 13/710,994

(22) Filed: Dec. 11, 2012

Related U.S. Application Data
(60) Provisional application No. 61/578,409, filed on Dec. 21, 2011.

(51) Int. Cl. C12Q 1/04

(52) U.S. Cl. CPC C12Q 1/04
USPC 435/288.7

(57) ABSTRACT

A combination assay device and method for simultaneously detecting the presence of hydrogen peroxide and D-lactic acid in vaginal fluid. The methods for detecting the hydrogen peroxide and the D-lactic acid are colorimetric-based. The device includes a pair of laminar flow substrates each having a solid-state compound for the detection of hydrogen peroxide and D-lactic acid disposed thereon. The solid-state format provides ease of use and storage.

This application claims priority to Provisional Patent Application No. 61/578,409, filed on Dec. 21, 2011. The entirety of Provisional Patent Application No. 61/578,409 is incorporated herein by reference.

### BACKGROUND

Vaginitis, a condition leading to irritation and inflammation of the vaginal area, is one of the leading reasons why women visit their physician. Bacterial vaginosis (BV) and yeast infections account for nearly 90% of all vaginitis cases, with BV being the most common. These conditions are characterized by the disruption of the normal “healthy” vaginal flora by invading pathogenic species, such as bacteria, viruses, protozoa, and fungus.

“Good” bacteria, such as lactobacillus (LB), are utilized to prevent pathogens from entering and disturbing the normal “healthy” vaginal flora and are the pre-dominant micro-organisms present in the vaginal flora in a healthy state. The vagina and LB flora are interdependent as the glycogen, produced by vaginal epithelial cells, acts as a source of nourishment for the bacteria while the LB flora provides an active defense against pathogenic species. More specifically, the LB flora produces D-lactic acid and hydrogen peroxide which lowers the pH in the vagina and prevents the growth of pathogenic bacteria, respectively. Therefore, in a normal “healthy” state, the vagina is acidic (low pH), contains high levels of D-lactic acid, and produces hydrogen peroxide.

When the LB flora is disrupted, abnormal and sometimes annoying vaginal symptoms occur causing many women to seek over-the-counter solutions. However, it has been reported that only one-third of women purchasing over-the-counter medications for vaginitis related symptoms correctly self-treat. Therefore, using over-the-counter products can actually worsen the problem.

There remains a need for an easy-to-use, inexpensive and reliable indicator that will allow women to routinely monitor their vaginal health. In addition, there remains a need for an indicator that is stable enough to be sold over the counter.

### SUMMARY OF THE INVENTION

The present invention relates to a method of making an indicator of vaginal health. A solid-state hydrogen peroxide indicator and a D-Lactic acid indicator are combined together in a single housing so that there is no fluid communication therebetween. The hydrogen peroxide indicator includes a substrate having a side on which an amount of horseradish peroxidase and an amount of TMB dye are disposed in a spaced apart configuration. The D-Lactic acid indicator includes a substrate having a side on which an amount of D-LDH, NAD⁺, diaphorase; and a NBT dye are disposed in a spaced apart configuration.

In another aspect of the invention there is a combination solid-state assay device for monitoring vaginal fluid. The device has a first substrate with a surface upon which NBT dye and a solid-state D-Lactic acid detection composition are disposed in a spaced apart configuration. The D-Lactic acid detection composition includes diaphorase, D-LDH, and NAD⁺. There is a second substrate which has a surface upon which TMB dye and a solid-state hydrogen peroxide detection composition are disposed in a spaced apart configuration. The second hydrogen peroxide detection composition is selected from horseradish peroxidase, potassium iodide, iodine and ammonium molybdate. The first and second substrates are located in a common housing where no fluid communication occurs between the first and second substrates. The first substrate may include a first conjugate pad located on the first substrate surface and spaced apart from the lactic acid detection composition, and a second conjugate pad located on the second substrate surface and spaced apart from the hydrogen peroxide detection composition.

Additional features and advantages of the present molecular system and homogeneous composition will be described in the following detailed description. It is understood that the foregoing general description and the following details description and examples are merely representative of the invention, and are intended to provide an overview for understanding the invention as claimed.

### BRIEF DESCRIPTION OF FIGURES

A full and enabling disclosure of the present invention, including the best mode hereof, directed to one of ordinary skill in the art, is set forth more particularly in the remainder of the specification, which makes reference to the appended figures in which:

FIG. 1 is a schematic diagram for the colorimetric detection of DLA;

FIG. 2 is a schematic diagram for the colorimetric detection of hydrogen peroxide based on ammonium molybdate;

FIG. 3 is a schematic diagram for the colorimetric detection of hydrogen peroxide based on iodine starch formulation;

FIG. 4 is a schematic diagram for the colorimetric detection of hydrogen peroxide based on horseradish peroxidase;

FIG. 5A is a side perspective view of one embodiment of an indicator according to the present disclosure;

FIG. 5B is a plan view of another embodiment of an indicator according to the present disclosure;

FIG. 6 is a chart depicting the activity of D-LDH as a function of time;

FIG. 7 is a chart depicting the activity of diaphorase as a function of time;

FIG. 8 is a colored photograph showing the stability of NBT from pH 4-9;

FIG. 9 is a chart depicting the stability of NBT from pH 4-9;

FIG. 10 is a chart depicting the effect of NAD⁺ concentration on the indicator response;

FIG. 11 is a chart depicting the effect of NBT concentration on the indicator response; and

FIG. 12 is a plan view of one embodiment of an housing for the assay device of the present invention.

### DETAILED DESCRIPTION OF THE INVENTION

The present disclosure relates to a diagnostic device that will allow women to routinely monitor their vaginal health. It is known that D-lactic acid (DLA) and hydrogen peroxide are produced by LB in order to prevent pathogenic micro-organisms from entering and disrupting the vaginal flora. When these levels decrease, an increased risk for infection occurs. Thus, by detecting the levels of both DLA and hydrogen peroxide in vaginal fluid, women can take control of their vaginal health. The diagnostic device of the present disclosure is capable of simultaneously and reliably detecting levels of DLA and hydrogen peroxide in vaginal fluid at concentrations of 1 mM and 1-20 μM, respectively.

The device uses two different compounds that, when in the presence of certain chemical reactions, can exhibit a color change detectable by the unaided eye. For a sample of vaginal fluid, one compound reacts to a decrease of hydrogen peroxide, and the other reacts to a decrease of DLA. Each compound corresponds to the two different detection methods used to determine the quantity or presence of hydrogen peroxide and DLA. Creating a device that can detect both DLA and hydrogen peroxide is challenging because each compound needs to be stable in the same environment, and each compound needs to be in a dry state, otherwise referred to as a solid state.

Desirably, the device of the present disclosure has an easy-to-handle format appropriate for use by consumers in non-clinical settings. Of course, the device may be desired by professionals, for example, at walk-in clinics that do not have elaborate test labs. The dry format also provides stability of each test component so that the indicator formulations will function when printed and immobilized on a solid substrate. In one aspect, the device employs an ink formulation that is printed onto lateral flow strips similar to that of a home pregnancy test. Suitable solid substrates for the lateral flow strips are in sheet form and include nonwoven materials as described herein.

The detection sensitivity of the device is different depending on whether it is DLA or hydrogen peroxide being detected. The DLA detection sensitivity is about 1 mM as this is the lowest level of DLA in vaginal fluid that is considered healthy. The hydrogen peroxide detection sensitivity is about 1 to about 20 μM as this range is considered to be in the healthy range for the level of hydrogen peroxide in vaginal fluid. In another aspect of the disclosure, a detection sensitivity of about 1 to about 10 μM or about 10 to about 20 μM may be appropriate.

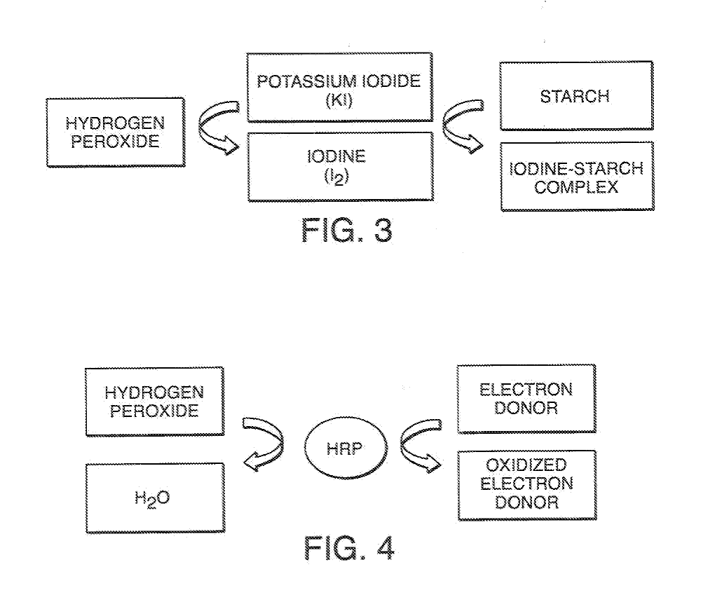

The most desirable method for detecting hydrogen peroxide is a colorimetric-based detection method capable of being converted from a solution-based format to a dry format. In one aspect of the disclosure, hydrogen peroxide is detected through a complexation with ammonium molybdate (FIG. 2), which yields an intense yellow response. In a second aspect of the disclosure (FIG. 3), hydrogen peroxide is detected through a complexation with iodine. Specifically, hydrogen peroxide causes the oxidation of potassium iodide (KI) to elemental iodine (I₂). If starch is present in the reaction, the hydrogen peroxide will complex with the iodine, yielding a blue response. In a third aspect of the disclosure (FIG. 4), a solution of horseradish peroxidase catalyzes the decomposition of hydrogen peroxide into water which in turn allows for the oxidation to tetramethylbenzidine (TMB, giving a bright green response. A wide array of electron donors such as ABTS (2,2′-azino-bis(3-ethylbenzthiazoline-6-sulphonic acid)), pyrogallol, guaiacol, o-dianisidine, leucomalachite green and other related electron donors can be used with this third detection method.

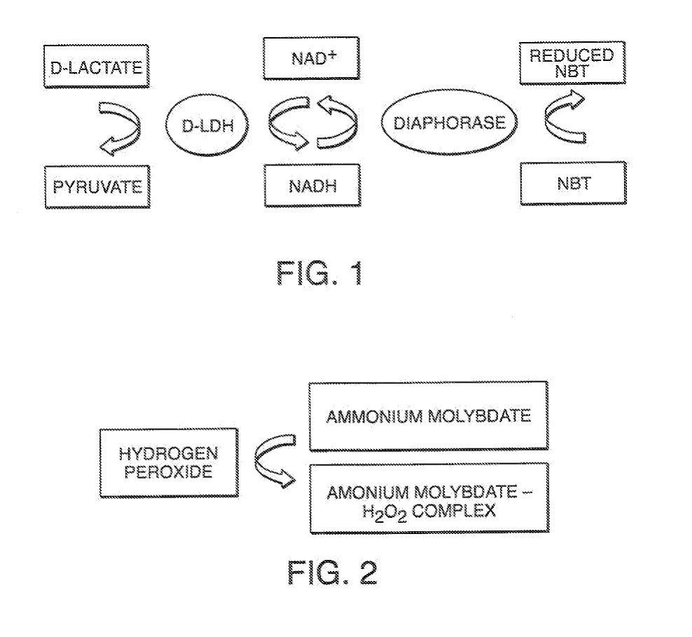

Referring to FIG. 1, the method for the detection of DLA is based on a two-step enzymatic process that causes the reduction of a dye leading to a colorimetric change. As this process requires multiple solid-state components, the formulation conditions are such that during storage, the enzymes will (1) retain an active state and (2) exhibit negligible dye degradation.

D-lactate dehydrogenase (D-LDH) catalyzes the conversion of D-lactate to pyruvate in the presence of the reduction of NAD+ to NADH. Diaphorase, a secondary enzyme, catalyzes the oxidation of NADH to NAD+ which in turn allows for the reduction of NBT (nitroblue tetrazolium), yielding a bluish purple solution. Suitable dyes for showing a response on an assay device include INT (p-iodonitrotetrazolium chloride), MTT (Dimethylthiazolyl-diphenyltetrazolium bromide), DCIP (2,6-Dichlorophenolindophenol). Other tetrazolium and tetrazolium-derived dyes may be utilized with this detection method as well.

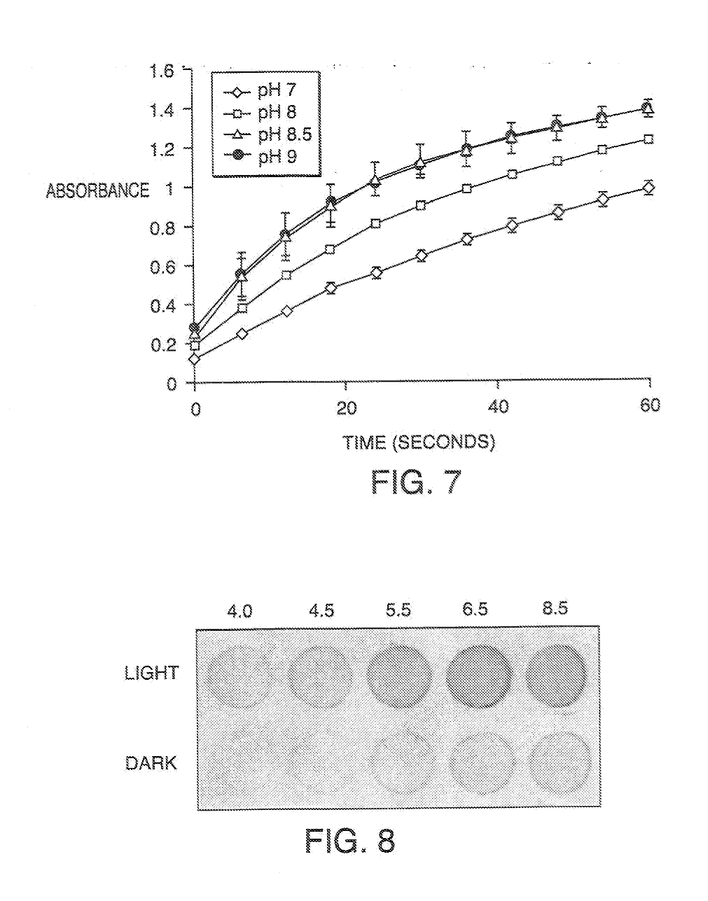

Before combining reagents D-LDH and diaphorase, the optimum pH for each component is determined to ensure reagent compatability. For D-LDH, at a temperature of 25 degrees C., the absorbance of light (λ=340 nm) can be monitored as a function of time for pH values ranging from pH 7-9 using a spectrophotometer such as that available from Varioskan (Thermo Electron Corporation, USA). As seen in FIG. 6, the maximum activity for D-LDH appears to be around a pH of 8.5. As seen in FIG. 7 for diaphorase, the absorbance of light (λ=560 nm) at a temperature of 25 degrees C. is monitored as a function of time for pH values ranging from pH 7-9. The optimum pH for diaphorase is about 8.5. Thus, both enzymes will function when constituted in a buffer of pH 8.5.

To assess whether NBT is stable under the same conditions as D-LDH and diaphorase, a NBT solution (10 μL of 1 mg/mL in buffer) is deposited on a solid substrate and either stored in light or dark environments to determine whether the dye degrades. As seen in FIG. 8, NBT begins to degrade above a pH of 5.5 for both light and dark conditions, resulting in a darker color. Therefore, it is most desirable to apply the indicator formulation in two steps as the stable pH ranges are different for the enzyme and the dye.

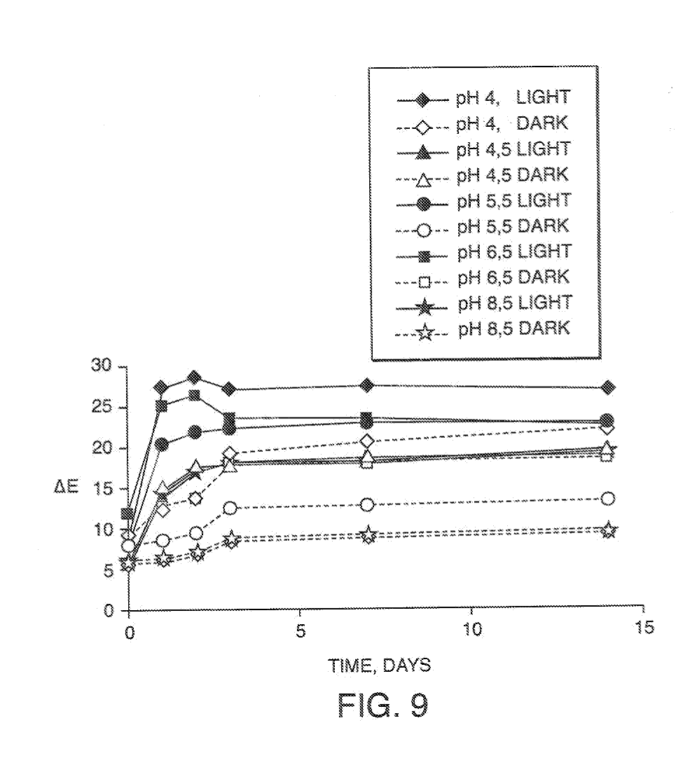

To quantify the results of the experiment shown in FIG. 8, ΔE values, which are the difference between the colors of the samples compared to the original unprinted substrates were considered. Referring to FIG. 9, the ΔE values were much lower for the coated substrates that were stored in the dark, which means that storing the indicator in the dark mitigates auto-reduction of the dye. As the pH went above 5.5, ΔE values steadily increased implying that the dye was degrading at these higher pH values. Therefore, it is most desirable to print NBT separately from the D-LDH/diaphorase formulation in order to retain activity. The photo-stability of the indicator is enhanced by refrigerating the indicator in an aluminum pouch at about 4 degrees C. to prevent the degradation and auto-reduction of NBT.

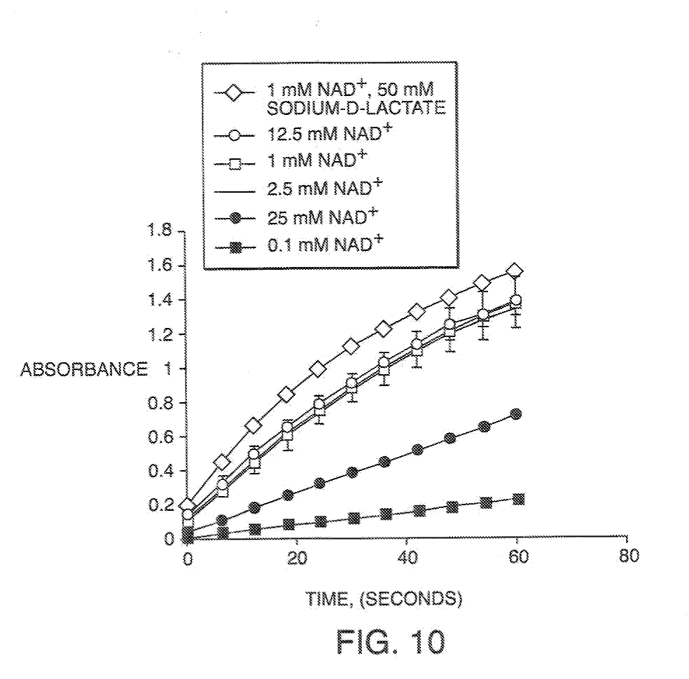

The concentration of NAD⁺ plays a role in the mechanism of detection as its oxidation leads to the reduction of the dye and therefore, the observed color response. Referring to FIG. 10, an enzyme/dye formulation was prepared with increasing concentrations of NAD⁺ to determine its effect on the reaction. When comparing the absorbance of light as a function of time, as the NAD⁺ concentration becomes too low or too excessive, the observed color response is diminished. Thus, the most desirable concentration of NAD⁺ is around 1 mM.

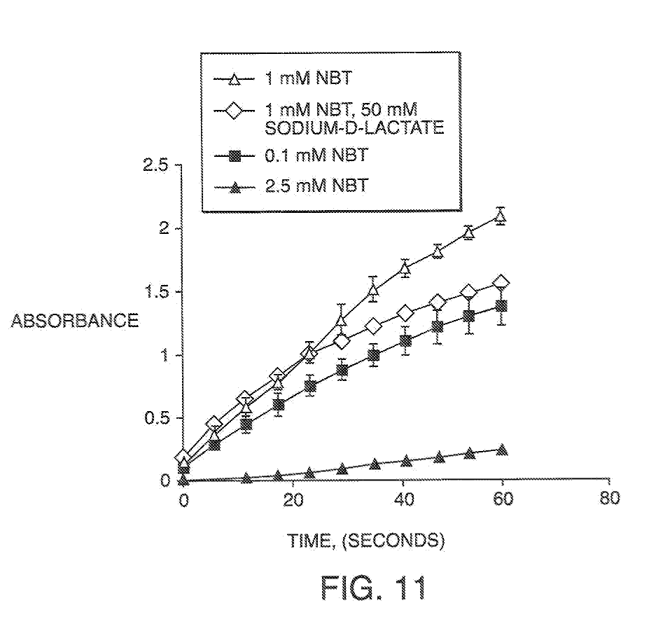

Likewise, the concentration of NBT plays a role in the indicator response. Referring to FIG. 11, to assess the effect of NBT concentration on the indicators function, enzyme/dye formulations were designed with increasing concentrations of NBT. The optimum concentration of NBT is approximately 1 mM, which is equal to the optimum concentration of NAD⁺.

### Assay Device

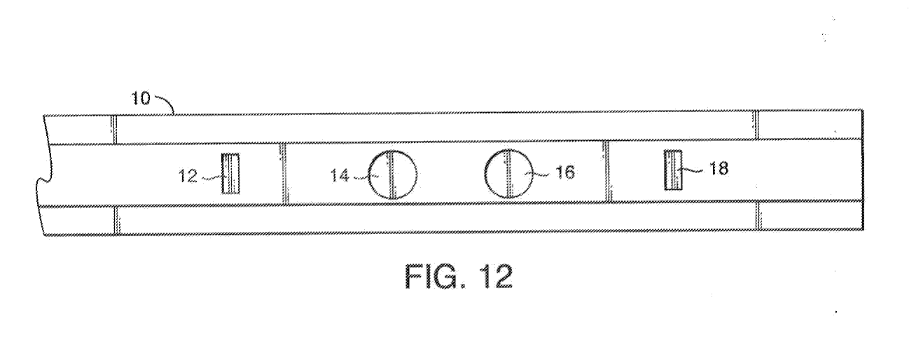

Referring now to FIG. 12, in one aspect of the disclosure, both indicator formulations are converted to indicators having a lateral flow format. Desirably, each separate lateral flow device is placed in a common housing 10 (e.g. a cardboard or plastic housing) to more conveniently provide a combination assay device capable of simultaneously detecting levels of hydrogen peroxide and DLA. The housing 10 has two adjacent lateral flow indicators housed therein, which are not in fluid communication. The indicators may be arranged in series as shown, or in parallel. There are two specimen application regions 14 and 16, where vaginal fluid may be deposited onto a conjugate pad, a sampling pad or a lateral-flow substrate, such as by a swab. Region 14 is associated with the hydrogen peroxide indicator, and region 16 is associated with the separate DLA indicator.

When a sample of vaginal fluid is applied to the combination assay device at regions 14 and 16, it causes a reaction in each lateral flow indicator. The test result relating to the detection of hydrogen peroxide appears in window 12. Similarly, the test result for DLA appears in window 18.

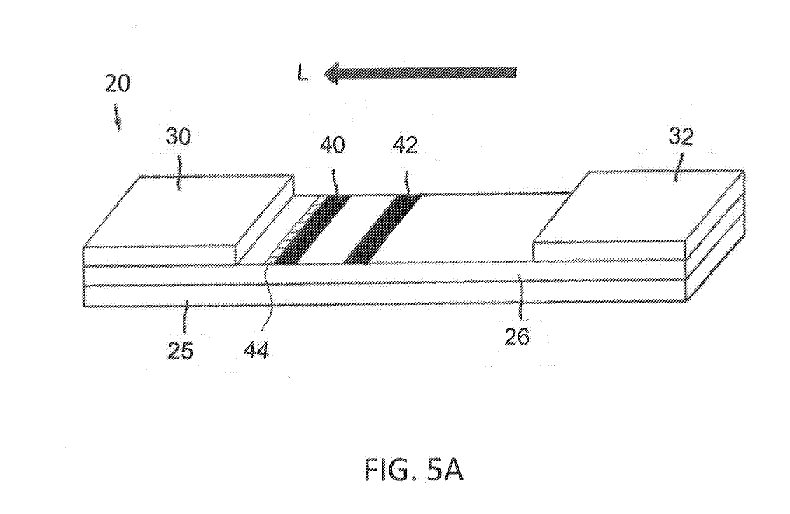

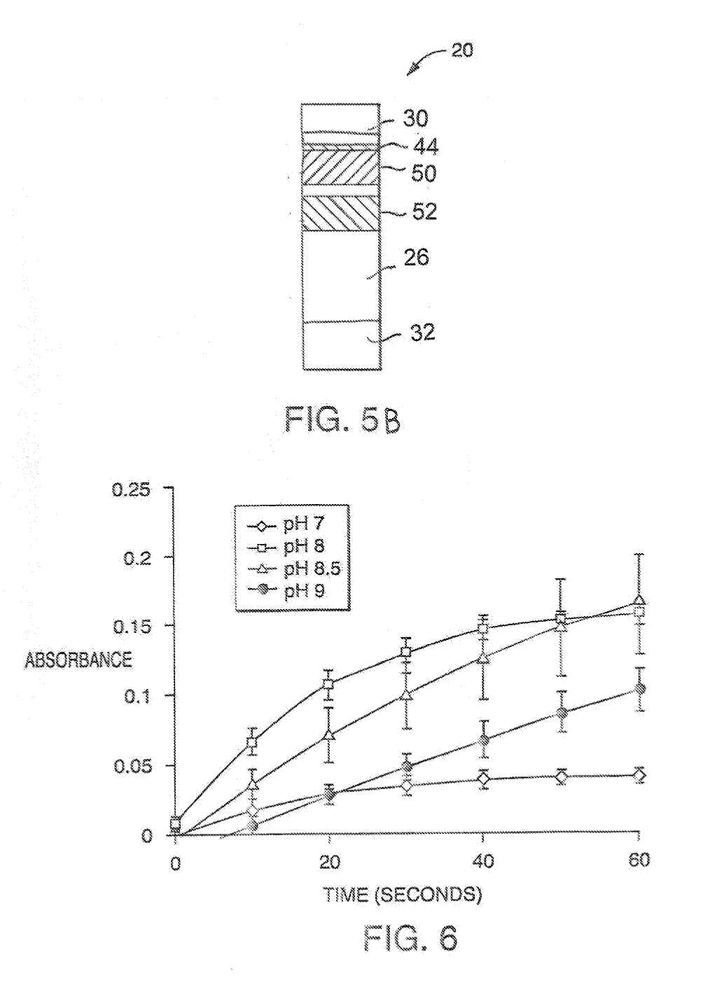

Referring to FIG. 5A, one embodiment of a membrane-based lateral flow-through device 20 that may be formed according to the present disclosure is described in more detail. As shown, the device 20 contains a porous membrane, substrate 26, which acts as a fluidic medium and is optionally supported by a rigid material 25. In general, the substrate 26 may be made from any of a variety of materials through which the test sample is capable of passing. For example, the materials used to form the susbstrate 26 may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO.sub.4, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In one particular embodiment, the susbstrate 26 is formed from nitrocellulose and/or polyether sulfone materials. It should be understood that the term “nitrocellulose” refers to nitric acid esters of cellulose, which may be nitrocellulose alone, or a mixed ester of nitric acid and other acids, such as aliphatic carboxylic acids having from 1 to 7 carbon atoms.

The device 20 may also contain an absorbent pad 30. The absorbent pad 30 generally receives fluid that has migrated through the entire susbstrate 26. As is well known in the art, the absorbent pad 30 may assist in promoting capillary action and fluid flow through the susbstrate 26.

To initiate the detection of an analyte within the test sample, a user may directly apply the test sample to a portion of the susbstrate 26 through which it may then travel. Alternatively, the test sample may first be applied to a sampling pad (not shown) that is in fluid communication with the susbstrate 26. Some suitable materials that may be used to form the sampling pad include, but are not limited to, nitrocellulose, cellulose, porous polyethylene pads, and glass fiber filter paper. If desired, the sampling pad may also contain one or more assay pretreatment reagents, either diffusively or non-diffusively attached thereto. In the illustrated embodiment, the test sample travels from the sampling pad (not shown) to a conjugate pad 32 that is placed in communication with one end of the sampling pad. The conjugate pad 32 is formed from a material through which the test sample is capable of passing. For example, in one embodiment, the conjugate pad 32 is formed from glass fibers. Although only one conjugate pad 32 is shown, it should be understood that other conjugate pads may also be used in the present disclosure. The direction of flow “L” is indicated by an arrow, and moves from the conjugate pad 32 to absorbent pad 30.

### DLA Indicator

The DLA indicator is prepared in a lateral flow format as shown in the assay device 20 of FIG. 5A. Specifically, the dye 40 formulation is prepared by dissolving NBT (17 mg/mL) in 4% PVA solution which is prepared in a sodium succinate buffer (25 mM, pH 4.5). An enzyme 42 formulation is prepared by dissolving NAD+ (43 mg/mL), diaphorase (17 mg/mL), D-LDH (0.29 mg/mL), PVA (2.70%) in a sodium succinate buffer (1 M, pH 8.5). A binder is added to the formulation to improve viscosity so that the formulation is more suitable for printing. One suitable binder is poly(vinyl alcohol) (PVA, Mn=27000).

The indicator for detecting DLA uses a hydrophobic boundary 44 to contain the dye 40 on the side located away from the reagent. To prepare the hydrophobic boundary, a solution of 5% polystyrene (PS) in xylene is applied to substrate 26 and dried. Desirably, the boundary 44 is abutted along one side of the dye 40, and is applied in layers (such as two to three layers). Using layers is desirable so that a sufficient hydrophobic barrier is present to keep indicator reagent within its region/zone on the indicator. The DLA indicator chemistry is applied adjacent the dried polystyrene solution using appropriate printing techniques. In the alternative, the DLA indicator chemistry is applied to the substrate 26, allowed to dry, and later masked with the 5% PS solution.

Suitable printing techniques for creating the hydrophobic boundary and the applying the indicator chemistry include ink jet, gravure and flexographic techniques. In addition, the hydrophobic boundary and indicator chemistries of the present disclosure may be disposed upon the indicator substrate by spraying or painting techniques.

The DLA indicator ink formulations are seen in Tables 1 and 2 located below. Desirably, several layers each of enzyme ink 42 and NBT dye 40 are printed onto substrate 26 in lateral stripes using a flexographic printing method or a comparable printing technique as noted above. Desirably, the stripes are spaced about 3-5 mm apart. Also, about nine layers of enzyme 42 and three layers of dye 40 are deposited on substrate 26. Multiple layers of the inks are desirable because it provides sufficient reagents on the substrate to see a color change visible to the human eye. Dilute solutions are prepared and printed multiple times to achieve necessary quantities because preparation of more concentrated solutions may lead to less stable solutions.

TABLE 1
Formulation for D-LDH/Diaphorase (Enzyme) Ink

| | Product Number | | | | |
|---|---|---|---|---|---|
| | and | Final | | Estimation* | |
| Reagent | Supplier | amount in ink | | of the amount/cm² | |
| NAD⁺ | N7004, Sigma | 24 | mg/ml | 78 | μg |
| Diaphorase | 4327, Worthington | 6 | mg/ml | 19 | mg |
| DLDH | E-DLDHLM, | 20 | μl/ml | 0.06 | μl |
| | Megazyme | | | | |

| | | |
|---|---|---|
| PVA | 26-88, Mowiol, | 1.60% |
| | Kuraray | |
| | America, Inc. | |
| Tris-HCl | | 50 mM, pH 8.5 |

TABLE 2
Formulation for NBT Ink

| | Product Number | | |
|---|---|---|---|
| | and | | Estimation* of the |
| Reagent | Supplier | Final amount in ink | amount/cm² |
| NBT | N6876, Sigma | 20 mg/ml | 6 μg |
| PVA | 26-88, Mowiol, | 4% | |
| | Kuraray | | |
| | America, Inc. | | |
| Succinate | | 25 mM, pH 4.5 | |
| buffer | | | |

### Hydrogen Peroxide Indicator

The hydrogen peroxide indicator has the same configuration as the assay device 20 of FIG. 5A except that enzyme 42 and dye 40 are replaced with other components. Referring to FIG. 5B, the TMB indicator dye 50 formulation may be prepared as an ink by dissolving TMB (140 mg) and ethyl acetate into a solvent such as dimethyl sulfoxide, wherein the ethyl acetate and dimethyl sulfoxide are in a 1:1 ratio. A thickener of PE9400 is ethanol increases viscosity of the formulation so that it has the adequate consistency to function as an ink. In one aspect, the TMB indicator dye formulation is prepared by dissolving TMB (140 mg) and ethyl acetate (3 mL) in a solvent such as toluene (0.7 g, 40 wt %).

The enzyme 52 ink may be prepared by dissolving peroxidase solution (500 μl of 10 mg/mL) in a 4% PVA solution in a phosphate buffer (2 mL, 0.1 M). The TMB indicator dye 50 may be applied to a substrate 26 (described herein) in one or more layers. Desirably, three layers of dye 50 are deposited onto substrate 26 using a printing or spraying technique.

Different embodiments of hydrogen peroxide indicator strips may be prepared using the two ink formulations set forth in Tables 3 and 4. Spaced apart lateral lines of selected TMB indicator dye 50 (A or B) and enzyme 52 formulations are printed onto a substrate 26 as described herein.

TABLE 3
Formulation for TMB Indictor Dye A

| | Product number | Final amount | Estimation* of the |
|---|---|---|---|
| Reagent | and producer | in ink | amount/cm² |
| TMB | Merck | 40 mg/ml | 12 μg |
| Ethyl acetate | J. T. Baker | NA | |
| | CAS 141-78-6 | | |
| PE9400 in | BASF | 80 mg/ml | 24 μg |
| toluene | | | |

TABLE 4
Formulation for TMB Indictor Dye B

| | Product number | Final amount in | Estimation* of the |
|---|---|---|---|
| Reagent | and producer | ink | amounts/test (2 layers) |
| Peroxidase | P8250, Sigma | 20 mg/ml | 4 μg |
| PVA | Mowiol, 26-88 | 3.20% | |
| | Kuraray | | |
| | America, Inc. | | |
| Phosphate | | 0.1M, pH 7 | |
| buffer | | | |

A lateral line of blocking material 44 such as PVA may be printed directly adjacent to the TMB dye 50 to prevent dispersion of active chemistry and enhance the color intensity. The line of blocking material 44 is placed between the absorbent pad 30 and dye 50, so that it abuts dye 50. Further, Bovine Serum Albumin (BSA), a protein known to block non-specific interactions, is printed under enzyme 42 to make the enzyme more accessible and in turn, enhance response time. The BSA line is of equal area to and is squared with the line of enzyme 42. Other large biological blockers may be used in lieu of BSA.

One method for monitoring vaginal flora includes the step of performing simultaneous diagnostic procedures to detect hydrogen peroxide and D-Lactic acid in vaginal fluid. For instance, the detection of D-Lactic acid is accomplished by using a solid-state compound set to (a) catalyze the conversion of D-Lactic acid to a pyruvate in the presence of a reduction of a solid-state D-LDH to NADH, and (b) catalyze the oxidation of NAD+ to NADH using a solid-state diaphorase; and by reducing a dye capable of a DLA color change when in the presence of D-Lactic acid. The diagnostic procedure for the detection of hydrogen peroxide is accomplished by contacting the vaginal fluid with a solid-state compound selected from the group consisting of horseradish peroxidase, potassium iodide, iodine and ammonium molybdate; wherein the solid-state compound is capable of an hydrogen peroxide color change in the presence of hydrogen peroxide. The simultaneous occurrence of the DLA color change and the hydrogen peroxide color change is indicative of a bacterial infection.

The present disclosure provides a relatively simple, compact and cost-efficient device for accurately detecting the desired analytes within vaginal fluid. The test result may be visible so that it is readily observed by the person performing the test in a prompt manner and under test conditions conducive to highly reliable and consistent test results. The device may then be discarded as a unit when the test is concluded.

The present disclosure may be better understood with reference to the following example.

### Example

Lab scale samples according to FIGS. 5A and 5B (minus the conjugate pad and the absorbent pads) were tested with healthy and unhealthy vaginal fluid. The tests were conducted at ambient temperatures and humidity.

After each reagent contacted an effective amount of vaginal fluid, the response time for the hydrogen peroxide indicator was about 0.5 minutes whereas the response time for the D-lactic acid indicator was about 1 minute. Within 2 minutes, the colored response of both indicators was clearly visible indicating that the sample contained “healthy” levels of D-lactic acid and hydrogen peroxide. When the indicator was treated with vaginal fluid samples that contained “unhealthy” levels of D-lactic acid (<1 mM) and hydrogen peroxide (<10-20 μM), no colored response was observed.

### Experimental Procedures

To acquire the activity assay for D-LDH, NAD+ (4.8 mM) and DLA (1.0 mM) is dissolved in Tris-HCl buffer (pH 7-9, 0.1 mM) to a final volume of 1 mL. The activity is assayed at 340 nm with a spectrophotometer (e.g a VARIOSKAN brand spectrophotometer available from Thermo Electron Corporation, USA). For a period of one minute and from a pH of 7 to 9, the absorbance is monitored as a function of time.

To acquire the activity assay for diaphorase; NBT (0.40 mM), diaphorase (12 nkat/mL), and NADH (0.47 mM) are dissolved in Tris-HCl buffer (50 mM, pH 7-9) to a final volume of 300 μL. The activity is assayed with the spectrophotometer at 560 nm. For a period of one minute and from a pH of 7 to 9, the absorbance is monitored as a function of time.

To determine the most desired pH of NBT, NBT (1 mg/mL) is dissolved in different buffers to achieve desired pH ranges:

pH 4-5.5, 50 mM sodium succinate buffer;
pH 6.5, 50 mM MES buffer (MES=2-[N-Morpholino]ethanesulfonic acid); and
pH 8.5, 50 mM Tris-HCl buffer. The buffered NBT solutions are deposited on a substrate, specifically filter paper, to form test spots (10 μl/spot). The test spots are stored in either light or dark environments and photographed after specified amount of time.

To assess the effect of NAD+ and NBT, assay solutions containing diaphorase (1 mg/mL; 5.6 U/mL), D-LDH (0.01 mg/mL; 4.25 U/mL), NAD+ (0.1-25 mM), NBT (0.1-2.5 mM), and D-lactic acid (5 mM; 50 mM) is dissolved in Tris-HCl (pH 8.5, 50 mM). The absorbance is measured by the spectrophotometer at 340 nm and/or 560 nm (the enzyme maximum absorbance).

The present invention has been described both generally and in detail by way of examples and the figures. Persons skilled in the art, however, can appreciate that the invention is not limited necessarily to the embodiments specifically disclosed, but that substitutions, modifications, and variations may be made to the present invention and its uses without departing from the spirit and scope of the invention. For example, it is contemplated that the assay device may be in dipstick form. Therefore, changes should be construed as included herein unless the modifications otherwise depart from the scope of the present invention as defined in the following claims.

1. The method of making an indicator of vaginal health comprising the steps of:
combining a solid-state hydrogen peroxide indicator and a D-Lactic acid indicator together in a single housing so that there is no fluid communication therebetween;
wherein the hydrogen peroxide indicator comprises a substrate having a side on which an amount of horseradish peroxidase and an amount of TMB dye are disposed in a spaced apart configuration;
wherein the D-Lactic acid indicator comprises a substrate having a side on which an amount of D-LDH, NAD⁺, diaphorase; and a NBT dye are disposed in a spaced apart configuration.

2. The method of claim 1 wherein the hydrogen peroxide indicator is configured to detect hydrogen peroxide at concentrations of 1 to 20 μM.

3. The method of claim 1 wherein the D-Lactic acid indicator is configured to detect D-Lactic acid at concentrations of about 1 mM.

4. The method of claim 1 further comprising the steps of dissolving the TMB dye in a 1:1 ratio of ethyl acetate and dimethyl sulfoxide; applying the dissolved TMB dye onto the substrate; and allowing the substrate to dry.

5. The method of claim 1 further comprising the step of dissolving the horseradish peroxidase in a PVA solution in a phosphate buffer.

6. A combination solid-state assay device for monitoring vaginal fluid, the device comprising:
a first substrate having a surface upon which NBT dye and a solid-state D-Lactic acid detection composition are disposed in a spaced apart configuration, wherein the D-Lactic acid detection composition comprises diaphorase, D-LDH, and NAD⁺; and
a second substrate having a surface upon which TMB dye and a solid-state hydrogen peroxide detection composition are disposed in a spaced apart configuration, wherein the second hydrogen peroxide detection composition is selected from the group consisting of horseradish peroxidase, potassium iodide, iodine and ammonium molybdate;
wherein the first and second substrates are located in a common housing where no fluid communication occurs between the first and second substrates.

7. The assay device of claim 6 wherein the diaphorase and the solid-state D-LDH are buffered to a pH of 8.5.

8. The assay device of claim 6 wherein the NBT dye is at a concentration of about 1 mM.

9. The assay device of claim 6 wherein the NAD⁻ is at a concentration of about 1 mM.

10. The assay device of claim 6 further comprising a hydrophobic boundary located on an edge of the NBT dye that is farthest from the detection composition.

11. The assay device of claim 6 further comprising a large biological blocker disposed under the solid-state hydrogen peroxide detection composition.

12. The assay device of claim 6 further comprising a blocking material located on an edge of the TMB dye that is farthest from the detection composition.

13. The assay device of claim 6 wherein the housing has windows for viewing the TMB dye and the NBT dye.

14. The assay device of claim 6 wherein the first substrate includes a first conjugate pad located on the surface and spaced apart from the lactic acid detection composition; and
wherein the second substrate includes a second conjugate pad located on the surface and spaced apart from the hydrogen peroxide detection composition.

15. The assay device of claim 6 wherein the hydrogen peroxide detection composition detects hydrogen peroxide at concentrations of 10 to 20 μM.
